(19) United States
(12) Patent Application Publication
(10) Pub. No.: US 2022/0143257 A1
(43) Pub. Date: May 12, 2022

(54) UVC STERILIZATION SYSTEMS AND METHODS FOR PATIENT VENTILATION

(71) Applicant: GE Precision Healthcare LLC, Wauwatosa, WI (US)

(72) Inventors: Russell J. Kuzelka, Madison, WI (US); Joseph J. Lacey, Madison, WI (US)

(73) Assignee: GE Precision Healthcare LLC, Wauwatosa, WI (US)

(21) Appl. No.: 17/094,505

(22) Filed: Nov. 10, 2020

(51) Int. Cl. A61L 9/20; A61M 16/20; A61M 16/00

(52) U.S. Cl. CPC A61L 9/20; A61M 16/20; A61M 16/009; A61L 2209/211; A61M 2205/3327; A61M 2230/43; A61M 16/0003

(57) ABSTRACT

A ventilator system includes a gas flow chamber configured to receive ventilation gas circulating in a ventilation gas pathway of the ventilator and at least one UVC lamp. The UVC lamp is configured to radiate UVC spectrum light into the gas flow chamber to inactivate pathogens in the ventilation gas. A flow sensor is configured to measure a gas flow rate of the ventilation gas and a controller is configured to receive the gas flow rate, determine an intensity based on the gas flow rate, and control power to the UVC lamp based on the intensity.

### FIELD

The present disclosure generally relates to patient ventilation systems, such as for anesthesia delivery and/or respiratory care in an intensive care unit, and more particularly to systems and methods for sterilizing ventilation gas within the ventilator.

### BACKGROUND

Ultraviolet light (UV) with wavelength shorter than 300 nanometer is extremely effective in killing microorganisms. The most potent or optimal wavelength range for damaging microorganism deoxyribonucleic acid (DNA) is approximately 254 nm-260 nm, with an effective sterilizing range within the “C” bandwidth of between 200 nm and 280 nm. This is called germicidal UV bandwidth or UVC. Ultraviolet light is not specific against selected bacteria and can be used to kill all pathogens with the use of slightly different doses.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

A ventilator system includes a gas flow chamber configured to receive ventilation gas circulating in a ventilation gas pathway of the ventilator and at least one UVC lamp. The UVC lamp is configured to radiate UVC spectrum light into the gas flow chamber to inactivate pathogens in the ventilation gas. In some embodiments, at least one flow sensor is configured to measure a gas flow rate of the ventilation gas and a controller is configured to receive the gas flow rate, determine an intensity based on the gas flow rate, and control power to the UVC lamp based on the intensity to achieve a specified UVC dose.

One embodiment of a system for sterilizing ventilation gas in a ventilator system includes a gas flow chamber configured to be positioned within an exhalation pathway of the ventilator system, such as between a patient and an exit port. The gas flow chamber is configured to receive exhalation gas exhaled by the patient. At least one UVC lamp is configured to radiate UVC spectrum light into the gas flow chamber to inactivate pathogens in the exhalation gas.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 depicts one embodiment of a ventilator system incorporating multiple UVC lamps in accordance with the present disclosure.

FIG. 2 depicts one embodiment of a system having at least one UVC lamp and configured for sterilizing ventilation gas in a ventilator.

FIG. 3 depicts one embodiment of a canister containing a plurality of UVC lamps and configured to sterilize ventilation gas in a ventilator.

FIG. 4 is another embodiment of a canister containing a plurality of UVC lamps configured for sterilizing ventilation gas in a ventilator.

FIG. 5 depicts another embodiment of a canister containing a plurality of UVC lamps and configured to sterilize ventilation gas in a ventilator.

FIGS. 6A-6D depict another embodiment of a canister containing a plurality of UVC lamps and configured for sterilizing ventilation gas in a ventilator, such as exhalation gas exhaled by a patient.

FIG. 7 depicts another embodiment of a canister configured to facilitate UVC radiation for sterilizing ventilation gas.

FIG. 8 depicts an embodiment of a system for sterilizing ventilation gas incorporated in a bellows within a ventilator system.

### DETAILED DESCRIPTION

The inventors have recognized increasing risks and costs associated with hospital-required infections as well as cross-contamination risks associated with medical equipment used on multiple patients. Based on the critical-care environments in which ventilators are used and susceptible patient population on which ventilators are utilized to support life—i.e., those who are immunocompromised, elderly, infants, and those with compromised respiratory systems—it is important that pathogenic or toxic microorganisms be eliminated from breathing system surfaces and ventilation gasses within the ventilator system.

The inventors have recognized that UVC light, or UVC energy, can be utilized to destroy the genetic material (DNA) of pathogenic microorganisms within the ventilator system and ventilation gas within the ventilator system, including to kill, or render non-viable, pathogens such as bacteria, fungal particles, mold spores, and viruses. Depending on the energy level of UVC delivered, it is possible to inactivate contagious microorganisms such as E. coli, Staphylococcus aureus, Mycobacterium tuberculosis bacterium and the Influenza, Rotavirus, Coronavirus, and Hepatitis A viruses. Many of these viruses are common in the healthcare setting and place the patient at risk for infection, lengthen the patient's stay, and increase cost both to the hospital and patient.

As disclosed herein, the inventors have developed UVC sterilization systems and methods for patient ventilation utilize UVC lamps, such as comprised of one or more UVC LEDs, to saturate areas within the ventilator breathing system with UVC light to destroy pathogenic or toxic microorganisms which may be resident within the ventilation gas, including gas that may be inhaled by the patient, exhaled by the patient, and/or drive gas that facilitates patient ventilation. In one embodiment, UVC wavelengths in the range of 200 nm to 280 nm is utilized at corresponding doses in order to destroy pathogens in the ventilation gasses. UVC wavelengths in the range of 207 nm to 220 nm is generally considered safe for exposure to human tissue, and the inventors have recognized that such wavelengths may be utilized in embodiments where human tissue may be exposed to the UVC light utilized for sterilization. In other embodiments, UVC light wavelengths of 260 nm may be utilized, which is generally considered a highly potent wavelength for disabling microorganisms. For example, one or more UVC lamps incorporating 260 nm UVC LEDs may be utilized to emit the UVC spectrum light.

The inventors have further recognized that the UVC lamps may be controlled based on values sensed within the ventilator system, including based on gas flow rates (such as gas flow rates within the patient ventilation circuit), moisture sensing, and/or the detection of volatile organic compounds (VOC) via one or more VOC sensors. For example, a controller may be configured to control power delivered to the UVC lamps based on sensed values, such as upon detection of VOCs and/or upon detection of a threshold amount of moisture. Alternatively or additionally, the power delivered to the one or more UVC lamps may be controlled based on gas flow rate in order to deliver a specified UV dosage. For example, for higher average patient circuit gas flowrates, the system may be configured to compensate by increasing power delivered to the one or more UVC lamps, thereby generating greater UVC intensity per area into the treatment field. The greater intensity thereby mitigates for the lower exposure time of a given volume of patient gas to the UVC light.

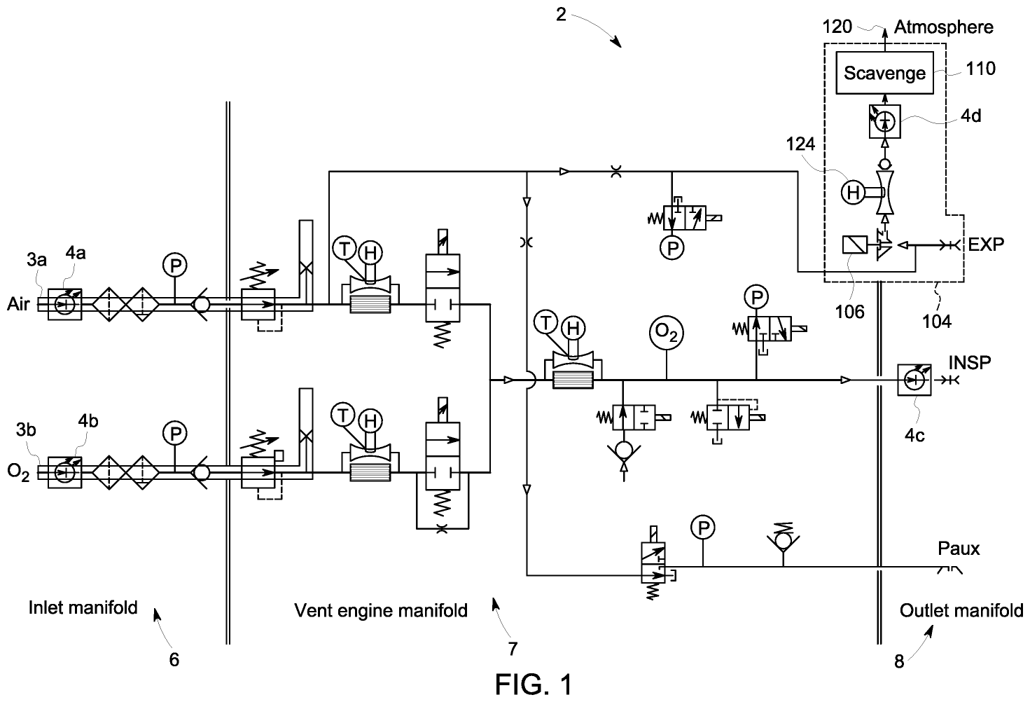

FIG. 1 depicts one embodiment of a ventilator system 2 configured to ventilate a patient from two gas sources, including an air gas source and an O₂ gas source. In other embodiments, fewer or additional gas sources may be used, including an anesthesia source. In various embodiments, the UVC module may be positioned within the inlet manifold system portion 6, the ventilator engine manifold 7, or the outlet manifold 8 of the ventilator system to sterilize the gases flowing therein. The depicted system 2 includes multiple UVC modules 4 positioned at various locations and configured to sterilize ventilation gas and/or surfaces within the ventilator system. Each UVC module 4 includes a gas flow chamber or cavity through which the ventilation gas flows—which could be inspiratory gases to be inhaled by the patient, expiratory gases exhaled by the patient, or a drive gas—and at least one UVC lamp configured to radiate UVC spectrum light into the flow chamber to kill pathogens in the ventilation gas. In certain embodiments, a UVC module 4 may be placed at the gas inlet 3a and 3b in order to sterilize gas exiting the gas source and provided to the inlet manifold 6. Alternatively or additionally, a UVC module 4 may be placed elsewhere in the inspiratory path of the ventilator between the gas source and the patient, such as at the outlet manifold 8. In other embodiments, the UVC module may be positioned within the vent engine manifold 7, such as at various locations within the ventilator pneumatics so as to sterilize gas flowing therein. In the depicted example, a first UVC module 4a is positioned at the primary gas inlet valve 3a, and thus between the gas source and the ventilator system 2. A second UVC module 4b is placed at or around the O₂ inlet valve 3b in order to sterilize the oxygen entering the ventilator system 2. A third UVC module 4c is placed in the inspiratory limb at the outlet manifold 8. In other embodiments, the UVC module 4 may be positioned in the exhalation flow path of the ventilator system 2 so as to sterilize the exhalation gases from the patient prior to venting the gases to atmosphere. For example, UVC module 4d is positioned in the exhalation flow assembly 104, and in the particular example between the exhalation valve 106 and the scavenging system 110.

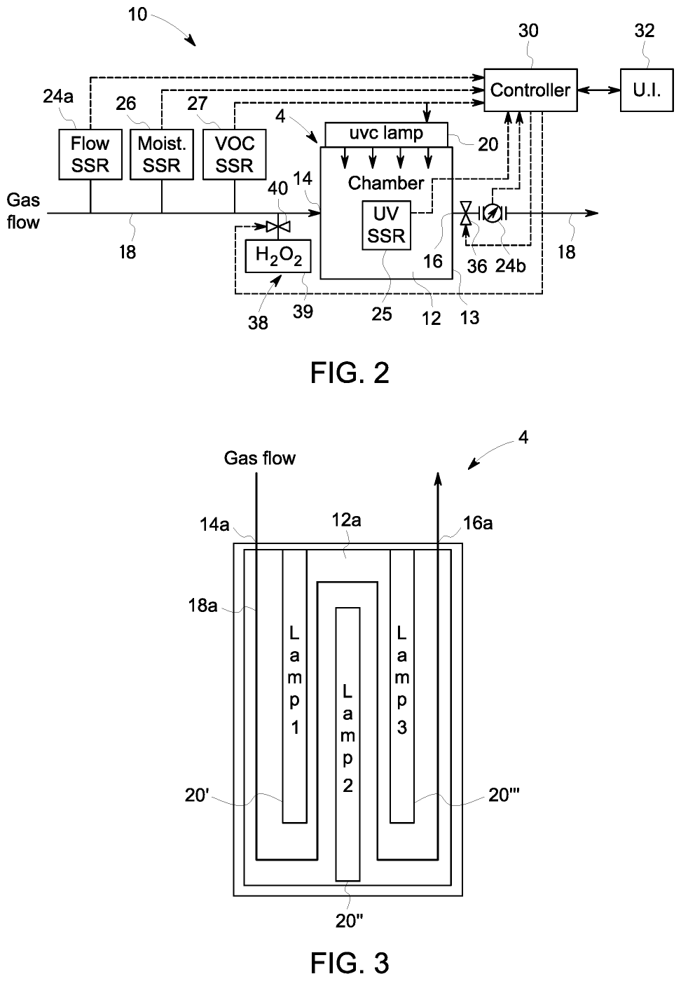

FIG. 2 depicts one embodiment of a sterilization system 10 configured to destroy pathogens in ventilation gasses within the ventilator system. Depending on the positioning of the sterilization system 10, it may be configured to receive and sterilize inhalation gasses to be delivered to the patient or exhalation gasses exhaled by the patient. In certain embodiments, the sterilization system 10 may be configured as a bi-directional device configured to receive and sterilize gas flow in the exhalation flow path and in the inhalation flow path.

The sterilization system 10 includes a UVC module 4 having an airflow chamber 12 positioned within the ventilation gas pathway within the ventilator system 2 and at least one UVC lamp 20 configured to radiate UVC light into the chamber 12. The airflow chamber 12 has an inlet port 14 and an outlet port 16, where the inlet port 14 receives gas along the gas flow path and the outlet port 16 expels gas, which then continues on the gas flow path 18 through the ventilator system and/or to be expelled from the ventilator system. A UVC lamp 20 is configured to radiate UVC spectrum light into the airflow chamber 12 to destroy pathogens in the ventilation gas within the chamber 12. For example, the UVC spectrum light may be configured to emit UVC bandwidth wavelengths, such as 260 nm wavelength. In various embodiments, examples of which are described herein, the UVC lamp 20 may be positioned on the edge of the chamber 12 or within the chamber 12. In certain embodiments, the chamber 12 may be configured to receive UVC radiation from multiple UVC lamps 20. For example, multiple UVC lamps 20 may be positioned around or within the chamber 12.

In certain embodiments, the sterilization system 10 may include a controller 30 configured to control power to the UVC lamp 20 in order to control the intensity of UVC light radiated into the chamber 12. The controller 30 is programmed to control the UVC lamp 20 based on one or more sensed values within the ventilator system 2. In one embodiment, the sterilization system 10 includes one or more flow sensors 24 configured to measure a flow rate of gas in the gas flow path 18. In the depicted embodiment, a flow sensor 24a is positioned on the gas flow path 18 upstream of the inlet port 14 to the chamber 12. A second flow sensor 24b is positioned downstream of the chamber 12, and in particular at or near the outlet port 16 such that it measures the flow rate of gas exiting the chamber 12. The controller 30 is configured to receive the flow rate measurements from each flow sensor 24a and 24b. In certain embodiments, the system may include only one flow sensor 24 providing flow rate information to the controller 30, which may be either upstream or downstream of the chamber 12 or situated within the chamber 12. The controller 30 may be configured to determine a UVC intensity based on the measured gas flow rate in order to achieve a UVC dosage. The degree to which the destruction of microorganisms occurs by UV radiation is directly related to the UV dosage. The UV dosage is calculated as:

D=I*t

where D is UV dose (mW s/cm²), I is intensity (mW/cm²), and t is exposure time (seconds).

When microorganisms are exposed to UV radiation, a constant fraction of the living population is inactivated during each progressive increment in time. This dose-response relationship for germicidal effect indicates that high intensity UVC energy over a short period of time would provide the same kill as lower intensity UV energy at a proportionally longer period of time. Therefore, for higher ventilator gas flow rates, the UVC dose could be increased accordingly, based on a control algorithm; improving efficiency and extending the life of the UVC lamp. The dosage is set based on the amount of UV radiation required to kill the desired pathogen. In certain embodiments, the controller 30 may store or access a table of dosages based on pathogens.

In certain embodiments, the system 10 may further include a user interface 32 configured to receive input from a user regarding dosage, and the user input device may be configured to facilitate such input in various ways. For example, the user interface 32 may be configured to receive a target pathogen from an operator and the system 10 may be configured to determine a dose based on the pathogen to be destroyed. In other embodiments, the user interface 32 may be configured to solicit and receive a dosage from the operator. The controller 30 then utilizes that dosage information to circulate an intensity and/or exposure time. In certain embodiments, the intensity of the UVC lamp 20 may be variable by the controller 30—namely, by varying the power to the UVC lamp 20. In other embodiments, the UVC lamp may have a fixed intensity. In certain embodiments, the system may include one or more valves 36 configured to control the flow rate within the chamber 12, and thus to vary the exposure time (t) in order to achieve a particular dose (D). For example, the valve 36 may be positioned at or near the outlet port 16 of the chamber 12 in order to control flow out of the chamber 12 and thereby control the amount of time that the ventilation gas is contained within the chamber and exposed to the UVC light. In various embodiments, the valve may be a PWM controlled proportional valve, or binary valve cycled intermittently. The controller 30 may be configured to control the valve 36 accordingly in order to achieve the desired the UVC dosage.

In certain embodiments, a UV sensor 25 may be configured to measure a UVC intensity within the gas flow chamber 12, which can be used as feedback for controlling the UVC lamp 20 and verifying achievement of the desired dose. Alternatively or additionally, the controller 30 may be configured to receive information from a moisture sensor 26 and/or a VOC sensor 27. For example, the moisture sensor 26 may be configured to sense moisture within the airflow chamber 12 or within the gas flow pathway 18 leading to the chamber 12. The controller 30 may be configured to control the UVC lamp based on the sensed moisture level so as to activate the UVC lamp and/or control its intensity based on the sensed moisture level. For example, the controller 30 may be configured to turn on the UVC lamp 20 when a threshold moisture level is detected. Similarly, the controller 30 may be configured to set an intensity level of the UVC lamp based on the moisture level measured by the moisture sensor 26, where the UVC lamp intensity is increased as the moisture level increases. Alternatively or additionally, the system 10 may include a VOC sensor 27 configured to sense the presence of organic compounds within the airflow chamber 12 and/or within the gas flow path 18 leading to the chamber 12. The controller 30 may be configured to control power to the UVC lamp 20 based on the detection of organic compounds so as to turn on the UVC lamp 20 and/or increase the intensity thereof when organic compounds are detected.

In certain embodiments, the sterilization system 10 may further include a hydrogen peroxide vaporizer 38 configured to vaporize hydrogen peroxide into the gas flow path entering the chamber 12. Liquid hydrogen peroxide in water is heated to produce a vapor of hydrogen peroxide and water, referred to as vaporized hydrogen peroxide (VHP). The temperature control is important, as the temperature will determine how much hydrogen peroxide/water can stay in a gas form without condensation. When in a gas form, hydrogen peroxide is typically used in the 0.1 mg to 10 mg/L range, which is very effective against microorganisms, including bacterial spores. 1 mg/L of hydrogen peroxide gas can kill 1 log of bacterial spores in about 1 minute (this time is called the D-value). As the concentration increases the microbicidal activity increases as well (e.g., the D-value at 10 mg/L is a few seconds). Hydrogen peroxide gas breaks down over time and on reaction with various surfaces turning into water and oxygen. The mechanism of cytotoxic activity is generally reported to be based on the production of highly reactive hydroxyl radicals from the interaction of the superoxide (O2.-) radical and H₂O₂ (O₂.—H₂O₂→O₂+OH—+OH.). The hydroxyl radical, OH., is the neutral form of the hydroxide ion (OH—). Hydroxyl radicals are highly reactive and consequently short-lived. Practically all organic compounds are attacked by OH.. The free radicals created by the attack of OH. on organic molecules will react further with O₂ or H₂O₂ in a chain reaction; therefore, several molecules of an organic substrate may be affected by the reaction sequence initiated by a single hydroxyl radical. The hydroxyl radical, .OH, is the neutral form of the hydroxide ion (OH—). Hydroxyl radicals are highly reactive and consequently short-lived. Most biological contaminants are deactivated by direct, uncatalyzed reaction with hydrogen peroxide (H₂O₂). Combining H₂O₂ vapor and concurrent irradiation with UVC light and reaction with catalytic surfaces, part of the H₂O₂ can be converted to hydroxyl radicals. Hydroxyl radicals are extremely reactive. Once the biological contaminant is dissolved in H₂O₂/H₂O, it will be rapidly degraded by reaction with OH., H₂O₂ and O₂. The time required for decontamination will largely be determined by mass transfer kinetics; specifically, by the rate of solution of the contaminant in the H₂O₂ vapor/liquid and/or in the H₂O₂/H₂O vapor condensing on catalytic surfaces.

The hydrogen peroxide vaporizer 38 may be positioned, for example, at the inlet of the chamber 12 such that the hydrogen peroxide mixes with the ventilation gasses flowing through the chamber and provides further sterilization thereof. For example, the hydrogen peroxide vaporizer may include a hydrogen peroxide container 39 and a dispensing valve 40 configured to inject the hydrogen peroxide vapor from the container 39 into the gas stream entering the chamber 12. A pressurized source of vaporized hydrogen peroxide (VHP), of around 30-35% concentration, is delivered into the chamber via a proportionally controlled valve or an injector valve, similar to an automotive style fuel injector. The VHP is then vented to scavenging along with the exhaled waste breath, in which case recapturing of the VHP would not be needed. However, the VHP can be recovered into H₂O₂ and H₂O using existing recovery technology currently employed in the state of the art. It is additionally envisioned that the system could utilize a VHP sensor to sense and regulate the concentration of VHP within the treatment volume, to target and maintain a user or facility specified concentration of VHP for the specific patient case.

In another embodiment, the waste gas from the patient can be bubbled through a volume of hydrogen peroxide with a low flow-resistance sparging filter. The sparging filter is used to generate microbubbles, increasing contact surface area of the waste gas for direct interaction with the liquid hydrogen peroxide. In some examples, efficient gas transfer and scrubbing/deactivation of the bacterial/viral load of exhaled patient gas is used to generate very high volumes of fine bubbles, such as bubbles having about a 1 mm diameter. It has been shown that a 1 mm bubble has 6 times the gas/liquid contact than that of a 6 mm bubble. Likewise, the container housing the liquid hydrogen peroxide solution and/or the UVC LED engines can utilize a catalytic surface coating such as silver to further enhance the rate at efficacy of de-activating biological/viral agents.

As described above, the gas flow chamber 12 may be positioned at various locations within the gas flow path of the ventilator, including within the inhalation flow path between the gas source and the patient, and/or in the exhalation flow path between the patient and discharging the gas to atmosphere. In certain embodiments, the sterilization system, including the chamber 12, may be integrated into the ventilator system 2. In other embodiments, the sterilization system 10 may be a standalone system or device that gets connected into the gas flow path, such as positioned between an exhalation valve at the patient end of the ventilation circuit and an exit port that releases the gas to atmosphere. Similarly, the sterilization system or standalone device may be positioned between the exhalation valve and a scavenging system configured to remove anesthetic agents from the exhalation gasses prior to releasing the gasses to atmosphere. In such embodiments, the sterilization system 10 is configured to sterilize the exhalation gasses from the patient before discharge to atmosphere. This prevents release of gasses containing dangerous pathogens, such as viruses, into the atmosphere which could then infect other people in the vicinity. For example, in an ICU setting the exhaled patient ventilation gasses are typically released into the atmosphere of the room in which the patient is being housed. The released gasses may contain viruses or other pathogens.

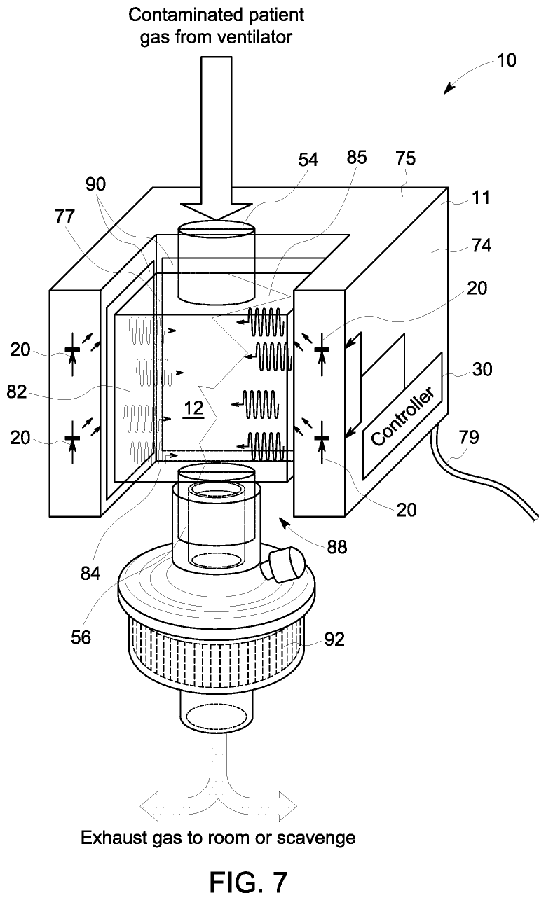

Currently, filters are sometimes used to remove such pathogens from the gasses vented to atmosphere. However, filters only provide a reduction in the total number of viable microbes per unit volume of gas and do not sufficiently eliminate such microbes to prevent transmission of infection. Further, certain microorganisms, such as viruses, can be as small as 0.02 microns and the filtering capabilities of such small organisms is limited. Further still, filter systems can become gross locations for microbes and thus can, in certain situations, exacerbate problems with pathogens. Utilization of UVC is a safer and more effective way to treat potentially contaminated waste gas from the patient prior to discharge into the patient's room other care area, thereby protecting caregivers and family members from exposure to potential viral and bacterial transmission. In certain embodiments such as that shown in FIG. 7, UVC may be used in conjunction with filtering to sterilize and filter the gas steam.

Alternatively or additionally, the sterilization system 10 can be positioned within the inhalation gas flow path in order to destroy pathogens within the inhalation gas prior to being inhaled by the patient. This can destroy molds, bacteria, or other pathogens that may have entered the inhalation gas from contaminated areas within the ventilator system 2. Utilization of UVC may reduce the risk of transmission of such pathogens to the patient to prevent causing infection, such as ventilator-induced pneumonia or other nosocomial infection. In still other embodiments, the sterilization system 10 may be utilized to treat specific areas of the ventilator where contamination may occur, such as contamination with mold and/or bacterial growth. This may particularly occur in areas where moisture tends to within the ventilator system 2.

FIGS. 3-5 depict various embodiments of UVC modules 4 comprising various chamber 12 and UVC lamp arrangements. As shown in the examples, the UVC module 4 may comprise any number of one or more UVC lamps 20 arranged around or within the chamber 12. The chamber 12 may be defined by a housing 42 having an inlet port 14 and an outlet port 16, wherein gas flows along a gas flow path 18 between the inlet and outlet. In certain embodiments, the module 4 may be configured to be bi-directional where the ventilator gas can also flow backward along the flow path 18 from the outlet 16 to the inlet 14. The amount of time that the gas spends in the chamber 12, between the inlet 14 and the outlet 16 is the dwell time (t).

The flow path 18 between the inlet 14 and outlet 16 ports may vary depending on the construction of the UVC module. In FIG. 3 the flow path 18a is a winding path around each of the UVC lamps 20′, 20″, and 20′″. In that embodiment, the lamps 20′, 20″, and 20′″ are situated in the chamber 12a, and the flow path 18a around each lamp so as to maximize UVC exposure time.

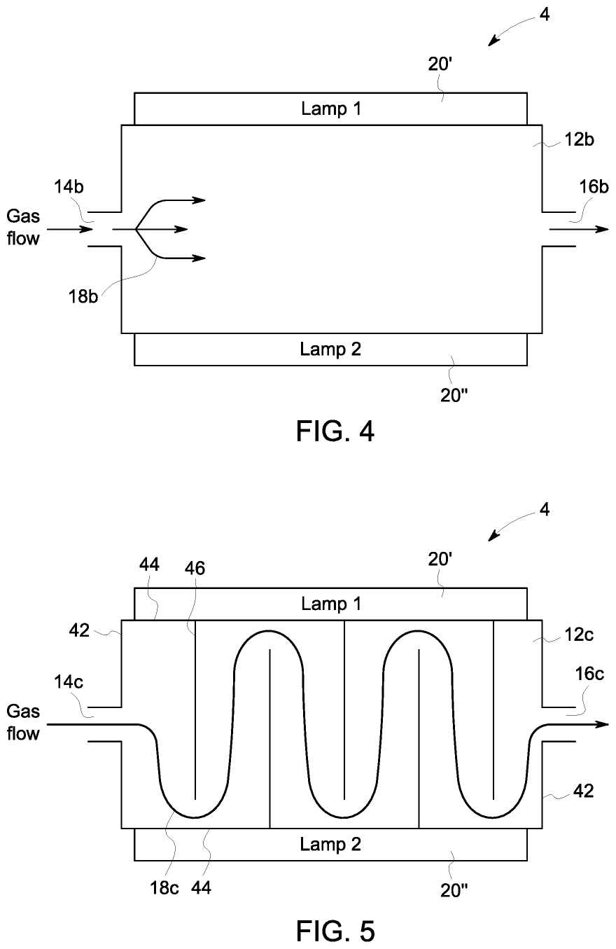

In the embodiment at FIG. 4, chamber 12b is an open chamber with two lamps 20′ and 20″ situated on opposing sides of the chamber 12b and configured to radiate UVC spectrum light into the chamber. Here, the gas flow path 18b between the inlet 14b and the outlet 16b is less structured within the open chamber volume flowing between the inlet 14b and the outlet 16b.

FIG. 5 depicts another embodiment of a UVC module 4. Two UVC lamps 20′, 20″ are positioned adjacent to the airflow chamber 12c which provides a circuitous flow path 18c back and forth across a width of the chamber 12c. This provides a defined flow path between the inlet port 14c and the outlet port 16c of the chamber 12c, which in some embodiments and applications may be beneficial for providing consistent and determinable exposure times based on measured flow rate. As can be seen from comparing the embodiments shown at FIGS. 3 and 5, the one or more UVC lamps 20 can be arranged in the flow path, as in FIG. 1, or surrounding the flow path, as in FIG. 5. In embodiments where the UVC lamps 20 are arranged and adjacent to the airflow chamber 12, UVC-transparent materials may be used to allow passage of UVC radiation through the housing 42 and into and throughout the chamber. For example, the airflow chamber 12 may be formed by a housing 42 having one or more windows 44 positioned adjacent to each lamp 20, 20′ to permit UVC radiation to travel through the housing 42 and into the airflow chamber 12c.

One or more dividers 46 may be positioned within the chamber 12c and configured to dictate the flow path 18c. The divider 46 may also be comprised of UVC-transparent material. For example, the windows 44 and/or dividers 46 may be comprised of quartz, which is UVC-transparent, or may be comprised of a polymer that is transparent to UVC. To provide just one example, the windows 44 and/or dividers 46 may be comprised of a clear, medical grade plastic with high UV transmission, such as cyclic olefin copolymer (COC). In certain embodiments, the remaining portions of the housing 42 may be comprised of UVC-opaque materials in order to contain the UVC radiation within the airflow chamber 12.

In certain embodiments, the sterilization system 10 may be all contained in a separate unit, or canister, that can be attached at certain points within the breathing circuit, such as those positions depicted in FIG. 1. For example, the canister 11 may be configured to attach at the output of the ventilation system where exhalation gasses from the patient are discharged to atmosphere. For example, the sterilization system 10 may be a self-contained canister 11 configured to attach within the exit assembly 104 of the ventilator system 2. In one example, the canister 11 is configured to be connected between an exhalation valve 106 and an exit port 120, or between the exhalation valve 106 and a scavenging system 110 (where present). As such, the canister is configured to sterilize the exhalation gasses from the patient before they are discharged to atmosphere. In one embodiment, the canister includes an integrated control 30 and power source 34. The power source 34 may be, for example, a battery integrated into the canister 11. In another embodiment, the canister may be configured to accept power such as via a power connection to the ventilator.

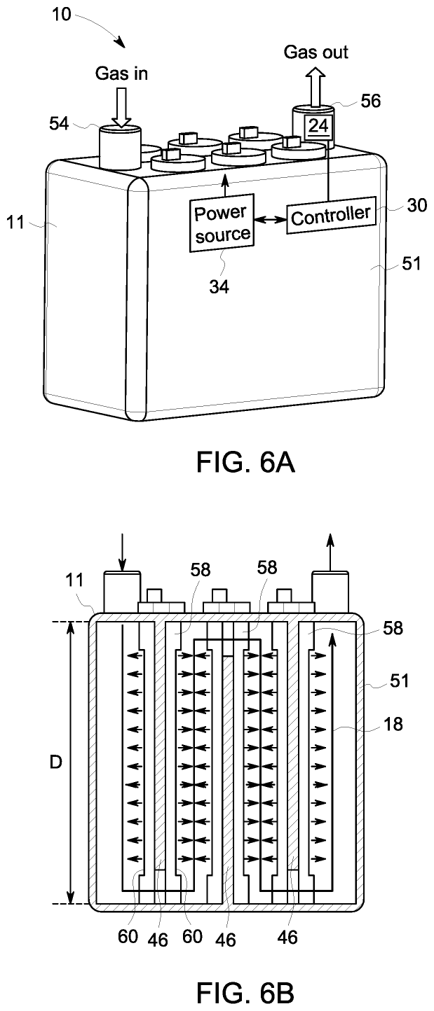

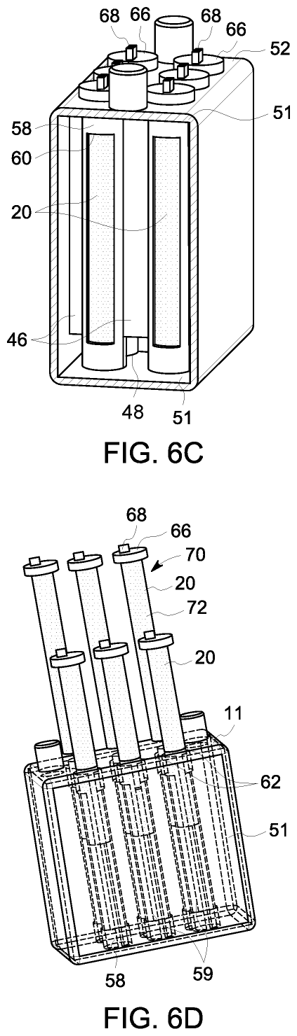

FIGS. 6A-6D depict one embodiment of a canister 11 that is separate, standalone unit, and configured for connection to the gas flow circuit of the ventilator 2. In certain embodiments, the canister 11 may be configured for single-patient use and may be a disposable unit that is replaced between uses of the ventilator system 2 with new patients, and/or when the sterilization system 10 embodied in the canister 11 fails or the battery dies, etc. In other embodiments, the canister 11 may be cleanable and usable and configured for use with multiple patients.

The canister includes a gas in the port 54 in the housing 51. The inlet port 54 is configured to connect to the flow path of the ventilator system, such as to be connected at or within the exhalation flow assembly, such as where the ventilator would vent to atmosphere and/or transfer gas to the scavenging system 110. The housing 51 has a gas outlet port 56 which may be configured to vent the sterilized gas to atmosphere and/or act connect to an inlet port of the scavenging system 110 to transfer the sterilized gas thereto. In an embodiment where the canister 11 is placed at the outlet of a ventilator system, the outlet port 56 may become the exit port 120 of the ventilator system where the exhalation gasses from the patient are vented to atmosphere.

The ventilation gasses, such as the exhalation gas exhaled by the patient, travel between the inlet port 54 and the outlet port 56 along a gas flow pathway 18. As described above, the gas flow pathway may take different forms depending on the instruction of the canister 11 and the airflow chamber 12 formed thereby. In the depicted example, the gas flow pathway 18 follows a switchback path across a depth D of the housing 51, thereby maximizing the pathway between the inlet and the outlet and providing maximum exposure to the plurality of UVC lamps housed in the canister 11.

FIGS. 6C and 6D depict one embodiment of the canister housing 51 having UVC receiving sections 58 configured to receive and hold a UVC lamp 20. In the depicted embodiments, the UVC receiving sections 58 are incorporated in or part of the dividers 46 such that the flow path 18 is guided past and around each of the UVC lamps 20 to maximize exposure. The UVC receiving section 58 may be configured to define a cavity 59 configured to securely hold the UVC lamp 20. The UVC receiving sections 58 have a shape that corresponds to that of the UVC lamp 20 in order to securely hold the UVC lamp 20 at a defined location within the airflow chamber 12. The UVC receiving section 58 is geared to hold the UVC lamp 20 in such a way that the UVC radiation is directed within the chamber. In one embodiment, the UVC receiving section 58 has one or more windows 60, such as a window on each side of the UVC receiving section 58 and positioned parallel to the flow path 19.

Each of the plurality of UVC lamps 20 may be removable from the canister 11, as is illustrated in FIG. 6D. The insertion port 62 facilitates insertion of a removable UVC lamp 20 into the UVC receiving section 58. In certain embodiments, the canister 11 may be configured to operate with a subset of the plurality of UCV lamps 20, and thus may operate with certain UVC receiving sections 58 unoccupied. In such embodiments, the canister 11 may include a plug or cap or other device for closing the insertion port 62 in the housing 51 when no UVC lamp 20 is in the receiving section 58. In the depicted example, each UVC lamp 20 has a top portion 66 configured to contact and/or fixable connect to a top side 52 of the housing 51. In certain embodiments, a handle 68 may extend from the top portion 66 to facilitate a user grabbing and removing the UVC lamp 20 from the UVC receiving section 58. In embodiments where the UVC module 4 embodied in the canister 11 does not have an integrated power source and/or integrated controller, the top portion 66 may provide a connection port through which the UVC lamp 20 is powered. In other embodiments, the canister 11 may include a battery, as described above.

UVC lamp 20 may include a lamp portion 70 housing a UVC light source and top portion 66 enabling connection to the housing 42. Each UVC lamp includes a UVC light source, such as one or more UVC LEDs. There are several other types of UVC capable sources commercially available such as low pressure mercury lamps, low pressure amalgam and medium pressure ultra violet (MPUV) lamps. Typically lamps are cylindrical lamps often with quartz sleeves for protection, although lamp shapes can be customized. For example, the UVC light source may be a 222 nm filtered far UVC excimer lamp. The lamp portion 70 includes a casing 72 surrounding the UVC LEDs or other UVC light source. The casing 72 provides optical functionality to facilitate radiation of the UVC spectrum light, such as having UVC-diffusing properties such that the casing acts as a diffuser to diffuse the UVC radiation throughout the chamber 12. In other embodiments, the casing 72 may be configured to focus the UVC light from the UVC light sources within the lamp 20, such as to focus UVC radiation at certain positions along the pathway 18.

As best shown in FIG. 6C, a cross sectional illustration of the canister 11, one or more dividers 46 may be provided to guide the flow path 18 (FIG. 6B) around each of the lamps 20. In the depicted example, the dividers 46 form passageways 48 along the outer ends of the Depth D of the airflow chamber 12. FIG. 7 depicts another embodiment of a canister 11 configured to connect with the gas flow circuit within a ventilator system 2, such as within an exhalation pathway between a patient being ventilated and an exhalation port where the exhalation gasses from a patient are vented to atmosphere. In the depicted example, the canister 11 includes a UVC module portion 74 and a chamber portion 76. The UVC module portion 74 houses one or more UVC lamps 20 configured to radiate UVC spectrum light into the chamber portion 76. The UVC module portion 74 has a module housing 75 configured to house the one or more UCV lamps 20, and may also be configured to house the controller 30. In the depicted example, the module housing 75 connects to a power cord 79 that receives power, such as from the ventilator system 2 in order to power the UVC lamps 20 through the controller 30. The controller 30 is configured to control power to the UVC lamps 20 as described herein.

The UVC module housing 75 is configured to hold the chamber housing 77, which in some embodiments is removable and replaceable. For example, the chamber housing 77 may be configured for single patient use such that the chamber housing 77 is disposed after use with each patient. In certain embodiments, the chamber housing 77 may be a cube or a rectangle with at least three sides in contact with the module housing 75 of the UVC module portion 74. Such side portions 82 of the chamber housing 77 may be formed of UVC-transparent material, examples of which are described above. The front side 84 and top side 85 may be formed of UVC-opaque material or otherwise have an outer casing the front side 84 and top side 85 to prevent the UVC light from leaving the chamber 12.

The chamber housing 77 may be configured to fit snuggly within a recess 88 in the module housing 75. The recess may comprise windows 90 in the module housing 75 to permit transmission of the UVC light from the lamps 20 to the chamber 12. The windows 90 are also comprised of UVC transparent material, examples of which are described above. Similar to the above-described embodiments, the system 10 exemplified in FIG. 7 is configured to receive ventilator gas, such as contaminated patient gas, from the ventilator at the inlet port 54. The gas is then maintained in the chamber 12 and exposure time (t) before it exits the outlet port 56 and eventually is ventilated to atmosphere and/or transferred to a scavenging system. In the depicted example, a filter 92 is positioned at the outlet port 56 to provide additional filtration prior to venting the exhalation gas to atmosphere. For example, the filter may be made from N96 filter media which work on the principles of inertial impaction, diffusion, and electrostatic attraction, where inertia impaction creates torturous paths such that it is difficult for 1 um particles and larger to have a straight flow path through the media. Diffusion filtration is for particles that are <1 um and works on creating path ways where the tiny particle continually move in random paths colliding with one another. In certain embodiments, the filter materials can be electrostatically charged to attract particles to the media fibers. Other filters such as HEPA filters can be used but care must be taken that trapped bacteria does not grow on the filter media.

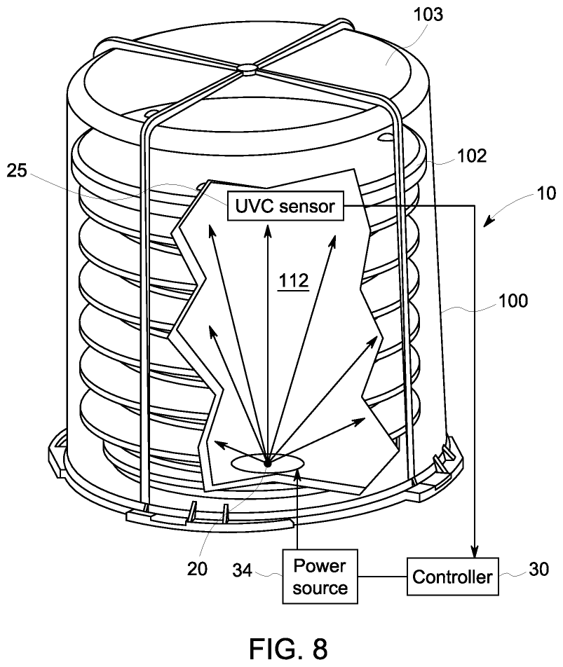

FIG. 8 depicts an embodiment of a sterilization system 10 configured to sterilize the gas flow chamber 112 which is the inside of a bellows 102 of a bellows system 100. The UVC lamp 20 is positioned within the bellows 102. The controller 30 controls power provided from the power source 34 to the UVC lamp 20 in order to control the intensity thereof. For example, the controller 30 may control the UVC light source 20 based on flow information provided by one or more flow sensors within the ventilator system 2. For instance, the controller 30 may receive a measured flow rate from a flow sensor in the exhalation path between the patient and the bellows system 100. Alternatively or additionally, the controller 30 may control the UVC lamp 20 based on ventilator rate, a breath period, breath volume, and/or other values related to the flow rate of exhalation gas from the patient to the bellows system 100. For example, such values may be provided from the operating controller of the ventilation system 2. In other embodiments, the ventilation system controller may operate as the controller 30 to perform the steps and functions described herein. In certain embodiments, a UV sensor 25 may be positioned within the bellows 102 to measure the UV intensity within the gas flow chamber 112 on the interior of the bellows. The measured UV can provide feedback to the controller 30 regarding the intensity, and thus the dosage, of UV being delivered.

The bellows system 100 comprises a bellows 102 that inflates and deflates within the cavity 103. When the bellows is in inflated, as shown in FIG. 8, the bellows expands within the cavity 103, such as expands upward as shown. As is standard in the art, the bellows inflates and draws gas from the patient to drive the exhalation portion of the patient's breath. In certain embodiments, the controller 30 may be configured to time the UVC intensity based on the inflation and deflation of the bellows, such as to turn on the UVC lamp and/or increase the intensity when the bellows is inflated and the cavity 103 is larger, and to decrease the intensity when the bellows is deflated. In another embodiment, the system 10 may be configured to perform a disinfection routine, such as during transition of an anesthesia ventilator system 2 or during a manual cleaning mode. In the disinfection routine, the system may be configured such that the bellows fully inflates and the UVC lamp is illuminated, such as to generate maximum intensity, for a period of time to reach a sterilization dosage. In another embodiment, the system 10 may be configured such that the UVC lamp 20 operates at a consistent intensity throughout the course of ventilation to continually provide UVC dosage within the interior of the bellows to perform a continual sterilization.

This written description uses examples to disclose the invention, including the best mode, and also to enable any person skilled in the art to make and use the invention. Certain terms have been used for brevity, clarity and understanding. No unnecessary limitations are to be inferred therefrom beyond the requirement of the prior art because such terms are used for descriptive purposes only and are intended to be broadly construed. The patentable scope of the invention is defined by the claims, and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

We claim:

1. A ventilator system comprising:
a gas flow chamber configured to receive ventilation gas circulating in a ventilation gas pathway of the ventilator;
at least one UVC lamp configured to radiate UVC spectrum light into the gas flow chamber to inactivate pathogens in the ventilation gas;
a flow sensor configured to sense a gas flow rate of the ventilation gas;
a controller configured to:
receive the gas flow rate;
determine an intensity based on the gas flow rate;
control power to the UVC lamp based on the intensity.

2. The ventilator system of claim 1, wherein the controller is further configured to determine the intensity based further on a UVC dosage set by an operator based on a pathogen to be inactivated.

3. The ventilator system of claim 2, further comprising an ultraviolet sensor configured to measure a UV intensity within the gas flow chamber, where the measured UV intensity provides feedback to the controller to confirm dose delivery.

4. The ventilator system of claim 1, wherein the airflow chamber is positioned between an exhalation valve and an exit port so as to inactivate pathogens in exhalation gases from a patient before discharge to atmosphere.

5. The ventilator system of claim 1, wherein the gas flow chamber is an interior of a bellows and the at least one UVC lamp is configured to radiate UVC spectrum light throughout the interior of the bellows.

6. The ventilator system of claim 1, wherein the airflow chamber is between a gas source and a patient and is configured to inactivate pathogens in inhalation gases to be inhaled by the patient.

7. The ventilator system of claim 1, further comprising:
a moisture sensor configured to sense moisture within the gas flow chamber or a volatile organic compound (VOC) sensor configured to sense a presence of VOCs within the gas flow chamber; and
a controller configured to control power to the UVC lamp based on the sensed moisture or the sensed presence of VOCs so as to increase an intensity of UVC when moisture or VOCs are detected.

8. A system for sterilizing ventilation gas in a ventilator system, the system comprising:
a gas flow chamber configured to be positioned within an exhalation pathway between a patient and an exit port, the gas flow chamber configured to receive exhalation gas exhaled by a patient; and
at least one UVC lamp configured to radiate UVC spectrum light into the gas flow chamber to inactivate pathogens in the exhalation gas.

9. The system of claim 8, further comprising a controller configured to:
receive a flow rate of the exhalation gas in the gas flow chamber;
determine an UVC intensity based on the gas flow rate and a UVC dosage; and
control power to the at least one UVC lamp based on the intensity.

10. The system of claim 9, wherein the controller is further configured to determine an average flow rate over a predetermined time period and to determine UVC intensity based on the average flow rate.

11. The system of claim 9, further comprising a flow sensor configured to measure an outlet flow rate at an outlet of the gas flow chamber, wherein the UVC intensity is determined based on the outlet flow rate.

12. The system of claim 9, wherein the flow rate is based on at least one of a ventilation rate, a breath period, and a breath volume of the patient such that the UVC intensity is determined based on the ventilation rate.

13. The system of claim 9, further comprising an ultraviolet sensor positioned within the gas flow chamber and configured to sense an actual UV intensity, wherein the controller is further configured to control the UVC intensity based on the actual UV intensity so as to reach the UVC dosage.

14. The system of claim 8, further comprising a hydrogen peroxide container and a dispense valve connected thereto configured to dispense vaporized hydrogen peroxide into the gas flow chamber.

15. The system of claim 14, wherein the dispense valve is configured to dispense vaporized hydrogen peroxide at an inlet of the gas flow chamber, further comprising a controller configured to control the dispense valve based on a flow rate measured at at least one of the inlet or an outlet of the gas flow chamber.

16. The system of claim 8, further comprising a canister with an internal pathway defining the gas flow chamber and housing the at least one UVC lamp such that the UVC lamp radiates the UVC spectrum light along the pathway.

17. The system of claim 16, wherein the canister is configured such that the at least one UVC lamp is removable from the canister.

18. The system of claim 17, wherein the canister is configured to removably receive a plurality of UVC lamps and is configured to operate with a subset of the plurality of UVC lamps in the canister.

19. The system of claim 18, further comprising a controller configured to:
determine a UVC intensity of each of the UVC lamps based on a number of UVC lamps in the canister and a UVC dosage; and
control power to each of the at least one UVC lamp based on the intensity.

20. The system of claim 16, wherein the canister is configured to be connected between an exhalation valve and an exit port or a scavenging system so as to inactivate pathogens in the exhalation gases from the patient before discharge to atmosphere.

21. The system of claim 8, wherein the gas flow chamber is within a bellows of a ventilator.
